(19) United States
(12) Patent Application Publication
(10) Pub. No.: US 2022/0346337 A1
(43) Pub. Date: Nov. 3, 2022

(54) Portulaca cultivar 'DPORMPZGL'

(71) Applicant: Danziger "DAN' Flower Farm, Beit Dagan (IL)

(72) Inventors: Pazit Sharoni, Beit Dagan (IL); Amir Zuker, Beit Dagan (IL); Gavriel Danziger, Beit Dagan (IL)

(73) Assignee: Danziger "Dan" Flower Farm, Beit Dagan (IL)

(21) Appl. No.: 17/246,084

(22) Filed: Apr. 30, 2021

(51) Int. Cl. A01H 6/00; A01H 5/10; A01H 5/02

(52) U.S. Cl. CPC A01H 6/00; A01H 5/10; A01H 5/02

(57) ABSTRACT

The invention relates to the field of Portulaca oleracea, specifically, the variety designated ‘DPORMPZGL’. The variety ‘DPORMPZGL’ is characterized by its controlled, semi-trailing plant habit, with free branching and vivid yellow flowers. The present invention relates to plant parts, including cells and any propagative material of the new variety ‘DPORMPZGL’, and use of any of the plant parts for reproducing the new variety ‘DPORMPZGL’. The present invention relates to methods using any plant parts of ‘DPORMPZGL’ for the purpose of deriving additional new Portulaca varieties. The present invention relates to seed, plants and plant parts produced by crossing ‘DPORMPZGL’ with any other Portulaca variety or other plant. The present invention also relates to methods to produce new varieties of Portulaca oleracea using the variety ‘DPORMPZGL’ and applying plant breeding techniques.

### FIELD AND BACKGROUND OF THE INVENTION

The present invention relates to a new variety of Portulaca oleracea referred to as ‘DPORMPZGL’ as well as to new, distinct and stable characteristics found in Portulaca oleracea. The present invention relates to plants which have all of the morphological and physiological characteristics described herein, as well as plant parts which can be used to reproduce plants having the characteristics specific to Portulaca oleracea referred to as ‘DPORMPZGL’. The present invention also relates to methods for producing these seeds and plants of Portulaca oleracea exhibiting the characteristics described herein. Furthermore, the present invention relates to a method of producing progeny Portulaca plants by crossing the Portulaca oleracea referred to as ‘DPORMPZGL’, as either the female or seed or male or pollen parent, with another Portulaca plant and selecting progeny. The present invention also relates to methods to produce new varieties of Portulaca oleracea using the variety ‘DPORMPZGL’ in a breeding program.

Portulaca is a member of the Portulacaceae family. Portulaca oleracea is a vascular land plant, with a nearly worldwide distribution. Portulaca oleracea is a succulent annual, widely used in the ornamental horticultural sector. Flowers are vibrantly colored and plants well suited for container and garden uses. This makes Portulaca oleracea an important target for the breeding of new varieties with novel characteristics. There is a demand for new varieties of Portulaca.

Asexual propagation of Portulaca can be performed by vegetative basal cuttings, however, propagation can also be performed by sowing seeds.

The new Portulaca cultivar is a product of a planned breeding program conducted by the inventors, Pazit Sharoni, Amir Zuker and Gavriel Danziger in Moshav Mishmar Hashiva, Israel. The objective of the breeding program was to produce useful new Portulaca varieties for ornamental horticultural production. The open-pollination resulting in this new variety was made during 2016.

A need exists for a greater variety of Portulaca cultivars with vigorous yet controlled plant habits. Larger flowers are also a desirable feature for the ornamental horticulture market.

A new Portulaca oleracea variety has been developed through a controlled breeding program and exhibits unique, desirable and stable characteristics. The Portulaca variety illustrated herein is referred to as ‘DPORMPZGL’.

The seed parent is the unpatented, propriety variety referred to as Portulaca PT-16-1356′. The pollen parent is unidentified. The new variety was discovered in July of 2017 by the inventor in a group of seedlings resulting from the 2016 open pollination, in a greenhouse in Moshav Mishmar Hashiva, Israel. After selection of the new variety, extensive confidential testing has been performed to determine and define the desirable characteristics of the new variety.

The new variety ‘DPORMPZGL’ can be produced by asexual reproduction to produce a population of progeny plants, each of which has the combination or characteristics as herein disclosed for the new variety.

### DEPOSIT INFORMATION

Biological material which can be used to produce the variety ‘DPORMPZGL’ is to be deposited with the National Collection of Industrial Food and Marine Bacteria (NCIMB), Ferguson Building, Bucksburn, Aberdeen, Scotland, a Budapest Treaty recognized depository which affords permanence of the deposit.

### OBJECTS OF THE INVENTION

The following embodiments and aspects thereof are described in conjunction with system, tools and methods which are meant to be exemplary, not limiting in scope

The present invention, in some embodiments thereof, relates to Portulaca oleracea plants and characteristics of these plants.

Portulaca oleracea, also known as ‘Purslane’, is valued as an ornamental flowering annual. Due to its ornamental value, attempts have been made to generate varieties of Portulaca oleracea incorporating novel and improved traits to the horticultural industry. Thus, whilst reducing the present invention to practice, the present inventors were able, to generate a unique variety of Portulaca oleracea.

The present invention provides Portulaca plant selections with controlled, semi-trailing plant habits, with free-branching. Flowers are large and vivid yellow and foliage is dark green. These characteristics in combination distinguish the new cultivar from typical Portulaca oleracea varieties.

These and other objectives have been achieved in accordance with the present invention which are the product of a planned breeding program conducted by the inventors. One embodiment of this invention is the Portulaca variety ‘DPORMPZGL’ described herein. The seed parent is the Portulaca oleracea line identified by code PT-16-1356 (unpatented).

Seeds which are variety ‘DPORMPZGL’ and contain the unique characteristic trait of acute flowering branch angles are to be deposited with the National Collection of Industrial Food and Marine Bacteria (NCIMB), Ferguson Building, Bucksburn, Aberdeen, Scotland.

Another embodiment relates to a plant produced from seeds which are Portulaca oleracea ‘DPORMPZGL’.

Another embodiment relates to a plant produced by vegetative means which are Portulaca oleracea ‘DPORMPZGL’. The present invention also relates to plant parts, such as pollen, seeds or inflorescence produced by Portulaca oleracea ‘DPORMPZGL’.

Another embodiment relates to a method of producing seed which are Portulaca oleracea ‘DPORMPZGL’.

Another embodiment also relates to a method of producing plants having all the physiological and morphological characteristics of the Portulaca oleracea ‘DPORMPZGL’. comprising the steps of (a) self-pollinating Portulaca oleracea ‘DPORMPZGL’. (b) harvesting seeds produced from said cross; and (c) producing plants from said harvested seeds.

The present invention also relates to producing progeny plants from the cross of Portulaca oleracea ‘DPORMPZGL’, as the female or male parent, with another Portulaca plant, and selecting progeny plants from this cross.

The present invention also relates to producing progeny plants of Portulaca oleracea ‘DPORMPZGL’, by any known means of vegetative propagation.

The present invention also relates to producing progeny plants of Portulaca oleracea ‘DPORMPZGL’, from natural or induced mutation.

Another embodiment relates to tissue culture produced from protoplast of cells from the Portulaca plant disclosed in the subject application, wherein said cells or protoplasts are produced from a plant part selected from the group consisting of pollen, ovules, embryos, protoplasts, meristematic cells, callus, leaves, anthers, cotyledons, hypcotyl, pistils, roots, root tips, flowers, seeds, petiole and stems.

Another embodiment relates to a plant or a part thereof, produced by growing Portulaca oleracea ‘DPORMPZGL’, wherein the plant part comprises at least one cell of Portulaca oleracea ‘DPORMPZGL’.

Another embodiment relates to tissue or cell culture of regenerable cells produced from the plants of Portulaca oleracea ‘DPORMPZGL’. This includes a Portulaca oleracea plant regenerated from the tissue or cell culture of Portulaca oleracea ‘DPORMPZGL’.

Another embodiment relates to a method of vegetatively propagating the plant Portulaca oleracea ‘DPORMPZGL’ comprising the steps of: collecting tissue or cells capable of being propagated from a plant of Portulaca oleracea ‘DPORMPZGL’; cultivating said tissue or cells to obtain proliferated shoots; and rooted said shoots to obtain rooted plantlets; or cultivating said tissue or cells to obtain shoots or to obtain plantlets and a plant produced by growing the plantlets or shoots of said plant.

A further embodiment relates to a method for developing a Portulaca oleracea plant in a Portulaca breeding program, comprising applying plant breeding techniques comprising crossing, recurrent selection, mutation breeding, wherein said mutation breeding selects for a mutation that is spontaneous or artificially induced, backcrossing, pedigree breeding, marker enhanced selection, haploid/double haploid production, or transformation to the Portulaca plant of Portulaca oleracea ‘DPORMPZGL’, or its parts, wherein application of said techniques results in development of an Portulaca oleracea plant.

A further embodiment relates to a method of introducing a mutation into the genome of Portulaca oleracea ‘DPORMPZGL’, and wherein the resulting plant comprises at least one genome mutation and producing plants therefrom.

In addition to the exemplary aspects and embodiments described above, further aspects and embodiments will become apparent by study of the following descriptions.

The cultivar ‘DPORMPZGL’ has not been observed under all possible environmental conditions. The phenotype may vary somewhat with variations in environment such as temperature, day length, and light intensity, without, however, any variance in genotype. The following traits have been repeatedly observed and are determined to be the unique characteristics of ‘DPORMPZGL’ These characteristics in combination distinguish ‘DPORMPZGL’ as a new and distinct Portulaca cultivar:

1. Controlled semi-trailing habit
2. Large flowers.
3. Vivid yellow flower color
3. Dark green foliage.
4. Freely branching habit

### Parent Comparison

Plants of the new cultivar ‘DPORMPZGL’ are similar to plants of the seed parent, in most horticultural characteristics, however, plants of the new cultivar ‘DPORMPZGL’ differ in the following;

1. Flowers of the new variety are yellow; flowers of the seed parent are dark orange with large yellow center.
2. Flower of the new variety are more open and nearly flat. Flowers of female parent is more conical to funnel-shaped.
3. Foliage of the new variety is darker than foliage of the seed parent

### Commercial Comparison

Plants of the new cultivar ‘DPORMPZGL’ can be compared to the commercial variety Portulaca Nano ‘Yellow’, unpatented. ‘These varieties are similar in most horticultural characteristics;

1. This comparator has more compact and dense habit than ‘DPORMPZGL’.
2. The flower color of this comparator is brighter yellow than flowers of ‘DPORMPZGL’.
3. Foliage color of this comparator is brighter green than foliage of ‘DPORMPZGL’.
4. Foliage of this comparator is larger than foliage of ‘DPORMPZGL’.

Plants of the new cultivar ‘DPORMPZGL’ can also be compared to the variety Portulaca ‘Cupcake yellow Chrome’, unpatented. These varieties are similar in most horticultural characteristics; however, ‘DPORMPZGL’ differs in the following:

1. Plant habit of ‘DPORMPZGL’ is semi-trailing, plant habit of this comparator is trailing.
2. ‘DPORMPZGL’ produces larger flowers than this comparator.
3. ‘DPORMPZGL’ produces yellow flowers; this comparator produces yellow flowers with orange venation.

### BRIEF DESCRIPTION OF THE FIGURES

The patent or application file contains at least one drawing executed in color. Copies of this patent or patent application publication with color drawings will be provided by the Office upon request and payment of the necessary fees.

The accompanying photographs in FIGS. 1 and 2 illustrate the overall appearance of the new Portulaca oleracea referred to as ‘DPORMPZGL’. Colors in the photographs may differ slightly from the color values cited in the detailed botanical description which accurately describes the color of ‘DPORMPZGL’.

FIG. 1 illustrates in full color a typical plant of ‘DPORMPZGL’ at about 8 weeks of age, grown in a 13 cm pot.

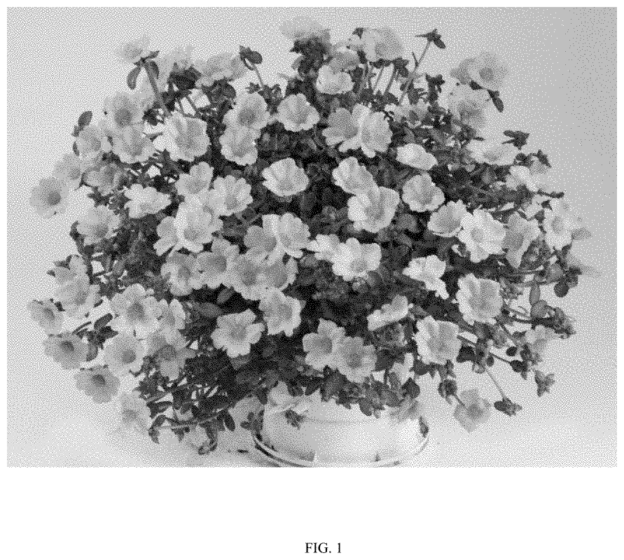

FIG. 2 illustrates in full color a close-up view of a typical flower of the new variety.

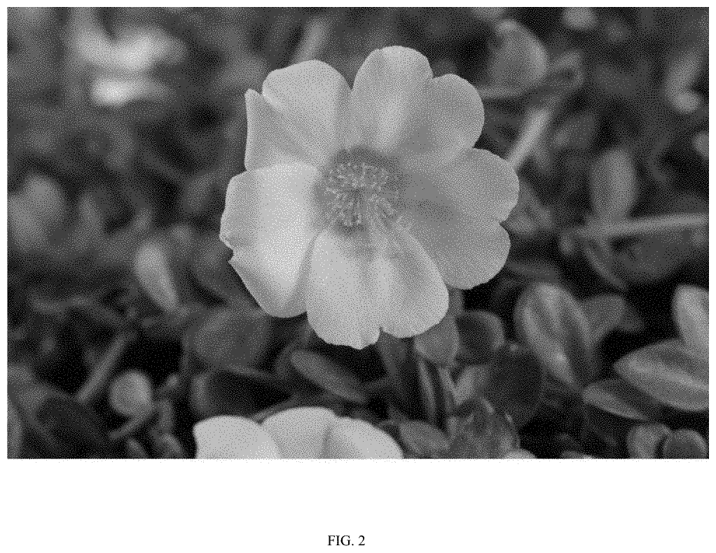

The photographs were taken using conventional techniques and although colors may appear different from actual colors due to light reflectance it is as accurate as possible by conventional photographic techniques.

### EXAMPLES

### Botanical Description

In the following description, color references are made to the Royal Horticultural Society Mini Colour Chart 2005 except where general terms of ordinary dictionary significance are used. The following observations and measurements describe ‘DPORMPZGL’ plants grown outdoors in Moshav Mishmar Hashiva, Israel, under natural lighting. Measurements were taken during Spring. The growing temperature ranged from approximately 24° C. to 38° C. during the days, 12° C. to 26° C. during the nights. Measurements and numerical values represent averages of typical plant types.

Botanical classification: Portulaca oleracea ‘DPORMPZGL’

### Propagation

Time to Initiate Rooting: Vegetative cuttings
Root Description: 10 to 12 days at approximately 24° C.
Root Description: Fine, fibrous; close to Yellow-White 158B in color, freely branching.

### Plant

### Growth Habit: Semi-trailing

Pot size of plant described: 13 cm.

### Height: About 16 cm.

Plant Spread: About 28 cm radius.
Growth Rate: Medium vigor

### Branching Characteristics:

### Length of Primary Lateral Branches: 26 cm.

### Diameter of Lateral Branches: 0.6 cm.

Quantity of Primary Lateral Branches: About 9 per 1 main stem

### Characteristics of Primary Lateral Branches:

Diameter: About 0.35 to 0.4 cm
Color: Tip Near Yellow-Green RHS 144B; Base near RHS Red-Purple 59B (Anthocyanin coloration)
Texture: Glabrous.
Strength: Moderately strong
   Internode length: Approximately 1.8 to 3 cm
   Age of Plant Described: Approximately 98 days from a rooted cutting.

### Foliage

### Leaf:

Arrangement: Alternate, simple
Quantity: Approximately 15 per branch.
Average Length: 2.5 cm.
Average Width: 1.2 to 1.6 cm.
Shape of blade: Obovate.
Apex: Rounded, slightly mucronate.
Base: Cuneate.
Margin: Entire
Texture of top surface: Smooth
Texture of bottom surface: Smooth
   Pubescence: glabrous, Succulent

Aspect: About 45-80°

### Color:

Young foliage upper side: Close RHS 137B
Young foliage under side: Close to RHS 138A
Mature foliage upper side: Close to RHS N137A
Mature foliage under side: Close to RHS 138A

Venation: Indistinguishable from leaf blade.

Petiole:
Length: About 1.2 mm
Diameter: About 1 mm
Color: Near RHS Yellow-Green 144B
Texture: Smooth, glabrous

### Flower

### Bloom Period:

Natural Season: Spring to Fall.

Days to flowering from rooted cutting: About 4 weeks to the first flowers.
Inflorescence and flower type and habit: Single flower, abundant flowering.
Rate of flower opening: About 3 days from bud to fully opened flower.
Flower Longevity on Plant: About 1-2 days.

### Persistent or Self-Cleaning: Self-Cleaning

### Bud:

Shape: Ovoid

Length: 1 to 1.2 cm

Diameter: 0.7 cm

Color: Near RHS Yellow-Green 144B; tip Near RHS Green 143B

### Flower Size:

Diameter: About 5 cm.

Depth: About 2 cm.

### Corolla/Petals:

Arrangement. 5 petals merged/fused at the base

Length: 2.5 cm.

Width: 2.1 to 2.3 cm.

Quantity: 5 petals

Texture: Smooth, glabrous

Apex: Emarginate to retuse

Shape: Obovate with a cuneate base

Margin: Entire

Aspect: Mostly flat, upper margin slightly rolled down.

Color: When opening:
Upper surface: RHS Yellow 9A
Lower surface: RHS Yellow 9B
Fully opened:
Upper surface: RHS Yellow 9A
Lower surface: RHS Yellow 9B

### Calyx/Sepals:

Quantity per flower: 2 Sepals per flower.

Shape: fused into a tubular calyx. Pointed apex.

Length: 0.9 to 1 cm

Width: 0.8 cm

Apex: Acute

Base: Fused

Margin: Entire

Texture: Glabrous

Color: Upper Surface: RHS Yellow-Green 144A
Lower Surface: close to RHS Yellow-Green 144B

### Peduncle: None

Fragrance: None detected.

### Reproductive Organs

### Androecium

Stamens:

Number: Many, about 60.

Filament length: Approximately 0.5 cm to 0.6 cm.

Anthers:
Shape: Oval
Length: About 0.6 mm
Color: Near RHS Yellow-Orange 14A
Pollen:
   Color: RHS Yellow-Orange 17A
   Quantity: Abundant

### Gynoecium

Pistil:

Number: 1

Length: 1.1 cm.

Style:
Length: 0.7 cm.
Color: Near RHS Yellow 9B

Stigma:
Shape: Six branched stigma
Color: Near RHS Yellow 9A
Ovary Color: Near RHS Yellow-Green 144A

### OTHER CHARACTERISTICS

Seeds and fruits: Seeds are minute and dark grey with a circular shape.
Disease/pest resistance: Neither resistance nor susceptibility observed. Portulaca is not typically susceptible to many diseases and pests. The most common diseases being typical root rots from over watering, or black stem, Dichotomophthora portulaca. The most common pest of Portulaca is Aphid.
Temperature tolerance: Will not tolerate temperatures below 7° C. High temperature tolerance near 40° C.

We claim:

1. Portulaca ‘DPORMPZGL’, representative biological material to be deposited at the NCIMB in Aberdeen, Scotland.

2. A plant or a plant part thereof produced by growing the plant of claim 1, wherein the plant or plant part comprises at least one cell of Portulaca ‘DPORMPZGL’.

3. A Portulaca plant or part thereof, having the physiological and morphological characteristics of the plants of claim 1.

4. The tissue or cell culture of regenerable cells produced from the plant of claim 1.

5. The tissue or cell culture of claim 4, comprising tissues or cells from a plant part selected from the group consisting of leaves, vegetative cuttings, pollen, embryos, cotyledons, hypocotyl, meristematic cells, roots, root tips, pistils, anthers, flowers and stems.

6. A method of producing Portulaca progeny comprising the steps of
(a) crossing a plant of Portulaca ‘DPORMPZGL’ as a female or male parent with another Portulaca plant or other plant and (b) selecting progeny.

7. The method according to claim 6, wherein the second plant is Portulaca ‘DPORMPZGL’.

8. A Portulaca seed that produces the Portulaca plants of claim 1.

9. A method for developing a Portulaca plant in a plant breeding program using plant breeding techniques, including crossing, recurrent selection, mutation breeding, wherein said mutation breeding selects for a mutation that is spontaneously or naturally induced or artificially induced, backcrossing, pedigree breeding, marker enhanced selection, haploid/double haploid production or transformation to a plant of Portulaca ‘DPORMPZGL’, or it's parts, wherein application of said techniques results in development of an Portulaca plant.
